(19) United States
(12) Patent Application Publication
(10) Pub. No.: US 2025/0000691 A1
(43) Pub. Date: Jan. 2, 2025

(54) PAIN RELIEF GARMENT SYSTEMS

(71) Applicant: Carla Jean Rudolph, Albuquerque, NM (US)

(72) Inventor: Carla Jean Rudolph, Albuquerque, NM (US)

(21) Appl. No.: 18/215,152

(22) Filed: Jun. 27, 2023

(51) Int. Cl. A61F 7/02; A41D 13/005; A61F 5/01

(52) U.S. Cl. CPC A61F 7/02; A41D 13/0058; A61F 5/0123; A61F 5/0127; A61F 5/013; A61F 2007/0238

(57) ABSTRACT

Thermal therapy systems comprising a compression garment that has an inner surface and an outer surface with one or more pockets, each having at least one opening and a circular pouch shape with an interior compartment functionally configured to receive and hold in place a thermal therapy pack over a target anatomical area, for example, the user's waist, a limb, a knee, ankle, elbow, or shoulder. The various embodiments of the present invention have the advantage that they can accommodate one or more hot/cold packs in compartments that can comfortably hold the hot/cold packs in place anywhere around the waist, a knee, ankle, elbow, or shoulder, and simultaneously provide a cloth barrier between the hot/cold packs and the person's skin.

### CROSS-REFERENCE TO RELATED APPLICATIONS

This application claims the benefit of U.S. Provisional Patent Application No. 63/420,499 filed on Oct. 28, 2022, which is incorporated by reference herein in its entirety. This application is also related to design patent application Ser. No. 29/895,780, filed Jun. 26, 2023, titled “WAISTBAND FOR PANTS” by Carla J. Rudolph, which is incorporated by reference herein in its entirety.

### FIELD OF THE INVENTION

Embodiments of the present invention relate generally to pain therapy systems. More particularly, embodiments of the present invention relate to pain thermal therapy compression garments.

### BACKGROUND OF THE INVENTION

Pain is a significant health concern affecting millions of individuals in the United States. It can range from mild discomfort to chronic and debilitating conditions, impacting both physical and mental well-being. An estimated 51 million adults in the US suffer from chronic pain. Furthermore, over 20 million Americans experience high-impact chronic pain, which significantly limits their daily activities.

Pain not only affects these individuals' lives but also has a substantial economic impact. It is estimated that the annual cost of pain in the US, including healthcare expenses and lost productivity, exceeds $600 billion dollars.

Hot and cold pack therapies are common non-pharmacological interventions for pain relief. Hot packs, such as heating pads or warm water bottles, provide soothing heat, while cold packs, such as ice packs, reduce inflammation and numb the affected area.

Hot pack therapy is effective for relieving muscle stiffness and promoting relaxation. It increases blood flow to the affected area, which can help reduce pain and promote healing. It has been found that hot pack therapy significantly reduced pain in patients with musculoskeletal disorders.

Cold pack therapy is beneficial for acute injuries, sprains, and post-surgical pain. Cold packs constrict blood vessels, reducing inflammation and swelling. They also numb the area, providing immediate pain relief. Cold pack therapy is recommended for the first 48 to 72 hours after an injury.

Alternating between hot and cold packs can provide additional benefits. This approach, known as contrast therapy, combines the advantages of both hot and cold therapies. It helps improve circulation, reduces muscle spasms, and promotes healing.

Hot/cold pack therapies are widely accepted due to their ease of use, affordability, and minimal side effects. Unlike medications, they do not carry the risk of addiction or adverse reactions. However, proper guidelines must be followed to avoid burns or frostbite, such as using a cloth barrier between the pack and the skin.

Hot/cold pack therapies can be effective for various pain conditions, including back pain, arthritis, migraines, and sports injuries. For example, it has been reported that hot pack therapy combined with exercise results in decreased pain and improved function in patients with chronic low back pain.

Hot/cold pack therapies can complement other pain management strategies. They can be used alongside medications, physical therapy, and other non-pharmacological approaches. Incorporating these therapies into a comprehensive pain management plan can enhance overall outcomes.

As the opioid crisis continues to pose a significant challenge, non-pharmacological interventions like hot/cold pack therapies play a vital role in promoting holistic and multimodal pain management strategies. They offer a safe, cost-effective, and easily accessible option for individuals seeking relief from pain.

However, hot/cold pack therapies often require individuals to stay still or hold the hot/cold packs in uncomfortable positions against their bodies. Holding the hot/cold packs in place with bandages, wraps, and/or strapping is difficult and/or uncomfortable and can result in painful rubbing against the skin, band marks, and/or unintended displacement of the hot/cold packs. The continuous need to apply hot/cold pack can significantly affect an individual's daily routine, preventing them from engaging in physical activities they enjoy, such as hiking, jogging, cycling, etc.

It would be extremely useful to have garments that can accommodate one or more hot/cold packs in compartments that can comfortably hold the hot/cold packs in place and simultaneously provide a cloth barrier between the hot/cold packs and the person's skin.

Accordingly, there remains a need in the art for a solution to at least one of the aforementioned problems.

### SUMMARY OF THE INVENTION

Embodiments of the present invention are directed to thermal therapy systems. In one embodiment, a system comprises a compression garment that has an inner surface and an outer surface comprising one or more pockets, and the one or more pockets comprise each at least one opening and a circular pouch shape with an interior compartment functionally configured to receive and hold in place a thermal therapy pack. Preferably, the thermal therapy pack is held over a target anatomical area, for example, the user's waist, a limb, a knee, ankle, elbow, or shoulder.

In one aspect the compression garment can be a pair of leggings. In a second aspect, the compression garment can be a long sleeve shirt.

In a different aspect, the thermal pack can be a hot pack or a cold pack. In another aspect, the thermal pack may be a hot/cold pack.

In another aspect, the thermal therapy system can have seam pockets with and without zippers to open and close the pockets.

In a different aspect, a thermal therapy garment system can comprise a compression garment that has an inner surface and an outer surface comprising one or more pockets, wherein one or more pockets comprise each at least one opening and a circular pouch shape with an interior compartment functionally configured to receive and hold in place a thermal therapy pack over a target anatomical area. For example, the user's waist, or a limb. In one aspect, the compression garment can be a pair of leggings.

In yet a different aspect, a thermal therapy garment system can comprise a pair of compression fabric leggings comprising an inner surface and an outer surface comprising a circular shape pouch pocket on the waist band, wherein the circular shape pouch pocket comprises a front opening and a rear opening and an interior compartment functionally configured to receive and hold in place a thermal therapy pack anywhere over the waist of a user.

The various embodiments of the present invention have the advantage that they can accommodate one or more hot/cold packs in compartments that can comfortably hold the hot/cold packs in place and simultaneously provide a cloth barrier between the hot/cold packs and the person's skin.

These and other objects, features, and advantages of the present invention will become more readily apparent from the attached drawings and the detailed description of the preferred embodiments, which follow and may be attained by means of the instrumentalities and combinations particularly pointed out in the appended claims.

### BRIEF DESCRIPTION OF THE DRAWINGS

The preferred embodiments of the invention will hereinafter be described in conjunction with the appended drawings, which are incorporated into and form a part of the specification and together with the description, are provided to illustrate and not to limit the invention, where like designations denote like elements, and in which:

FIG. 1 presents a front view of an embodiment of the invention for a thermal therapy system comprising compression leggings with a waistband circular pouch shaped 360 degree pocket with a front and a rear opening;

FIG. 2 presents a rear view of the embodiment of FIG. 1;

FIG. 3 presents a front view of an embodiment of the invention for a thermal therapy system comprising compression leggings with a circular pouch shaped 360 degree pocket with a front and a rear opening on each knee and ankle;

FIG. 4 presents a rear view of the embodiment of FIG. 3;

FIG. 5 presents a front view of an embodiment of the invention for a thermal therapy system comprising a compression long sleeve shirt with elbow and shoulder circular pouch shaped 360 degree pockets with a front opening on each elbow and a rear opening on each shoulder (see FIG. 6); and

FIG. 6 presents a rear view of the embodiment of FIG. 5.

Like reference numerals refer to like parts throughout the several views of the drawings.

### DETAILED DESCRIPTION

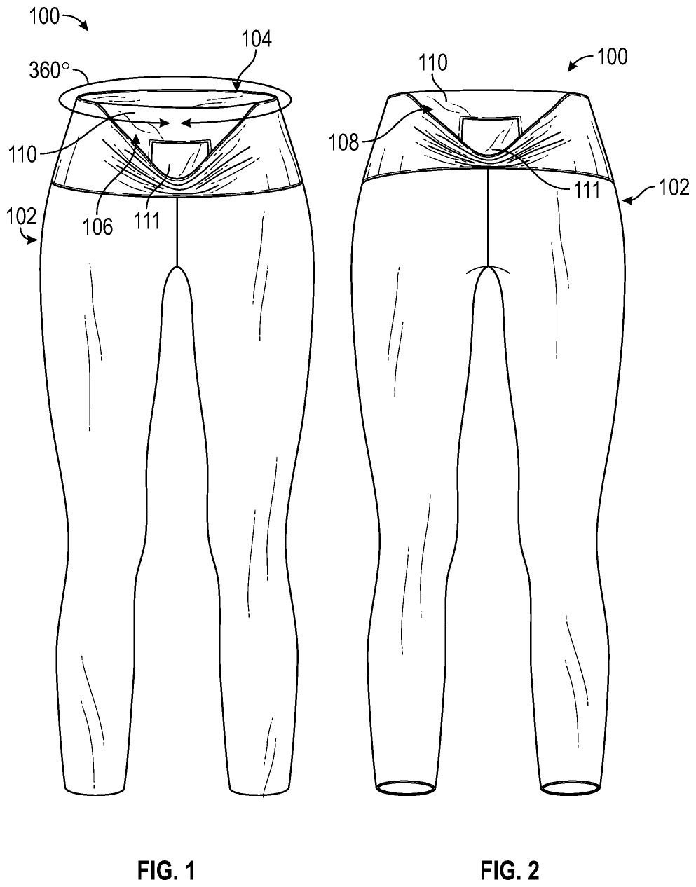

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein-as understood by the ordinary artisan based on the contextual use of such term-differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The term “comprising” can also encompass the terms “consisting essentially of” and “consisting of.” Note that in the specification and claims, if any, “about” or “approximately” means within twenty percent (20%) of the numerical amount cited.

As used throughout the specification and claims, the term “seam pocket” means the type of pocket in which the opening falls along the seam line of a garment where two fabric panels overlap and can include in-seam pockets.

Shown throughout the figures, embodiments of the present invention are directed to thermal therapy systems. Referring to FIGS. 1-2, a thermal therapy system 100 comprises a pair of compression leggings 102 with a waistband circular pouch shaped pocket 104. In one embodiment the waistband circular pouch shaped pocket 104 covers the whole waist of the user (i.e., goes 360° around). In one embodiment, the waistband circular pouch shaped pocket 104 comprises at least one opening, for example, a front opening 106, and a rear opening 108 and has a compartment 110 functionally configured to receive and hold in place a thermal therapy device 111, for example, a hot pack, a cold pack, and/or a hot/cold pack or combinations thereof. As denoted by the 360° arrows on the waist area of the leggings 102 in FIG. 1, the versatility of the waistband circular pouch shaped pocket 104 allows a user to selectively position one or more thermal therapy devices in the compartment 110 anywhere around the waist of the user.

In one embodiment, the front opening 106 and the rear opening 108 are seam pockets. In one embodiment the front opening 106 and the rear opening 108 comprise zippers to open and close the compartment 110.

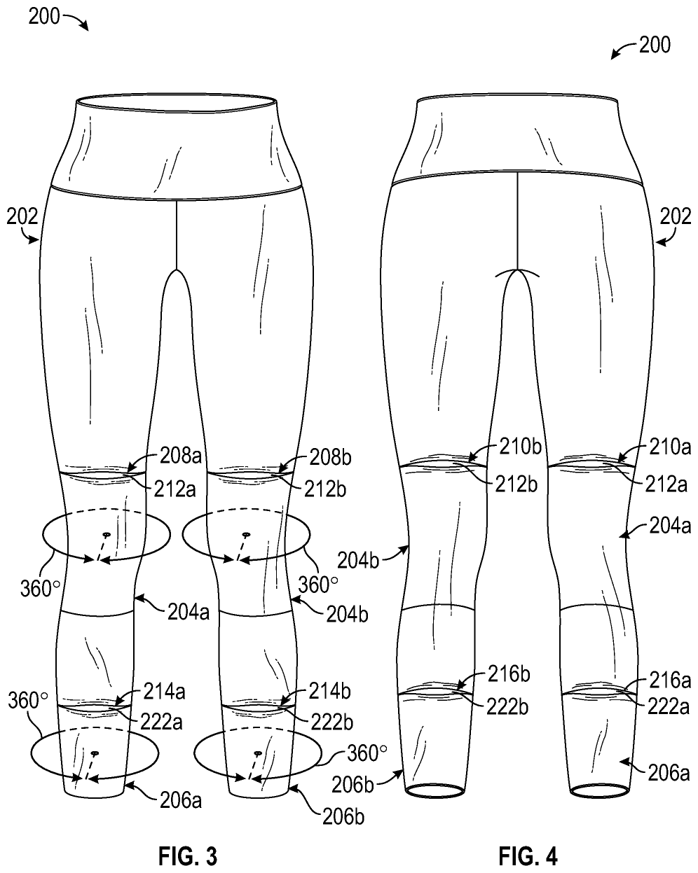

Referring now to FIGS. 3-4, in a different embodiment, a thermal therapy system 200 comprises a pair of compression leggings 202 with two knee circular pouch shaped pockets 204a and 204b. In one embodiment, the knee circular pouch shaped pocket 204a comprises a front opening 208a and a rear opening 210a to open and close a compartment 212a, and the knee circular pouch shaped pocket 204b comprises a front opening 208b and a rear opening 210b to open and close a compartment 212b. In one embodiment, the compartments 212a and 212b are preferably functionally configured to receive and hold in place a thermal therapy device (not shown, but similar to the thermal therapy device 111 of FIG. 1), for example, a hot pack, a cold pack, and/or a hot/cold pack or combinations thereof. As denoted by the 360° arrows around the knee areas of the leggings 202 in FIG. 3, the versatility of the 360° knee circular pouch shaped pockets 204a and 204b allows a user to selectively position one or more thermal therapy devices in the compartments 212a and 212b anywhere around the knee of the user.

In one embodiment, the front openings 208a and 208b, and the rear openings 210a and 210b are seam pockets. In one embodiment the front openings 208a and 208b, and the rear openings 210a and 210b comprise zippers to open and close the compartments 212a and 212b.

In one embodiment, the thermal therapy system 200 also comprises two ankle circular pouch shaped pockets 206a and 206b. In one embodiment, the ankle circular pouch shaped pocket 206a comprises a front opening 214a and a rear opening 216a to open and close a compartment 222a, and the knee circular pouch shaped pocket 206b comprises a front opening 216b and a rear opening 214b to open and close a compartment 222b. In one embodiment, the compartments 222a and 222b are preferably functionally configured to receive and hold in place a thermal therapy device (not shown), for example, a hot pack, a cold pack, and/or a hot/cold pack or combinations thereof. As denoted by the 360° arrows around the ankle areas of the leggings 202 in FIG. 3, the versatility of the 360° ankle circular pouch shaped pockets 206a and 206b allows a user to selectively position one or more thermal therapy devices in the compartments 222a and 222b anywhere around the ankle of the user.

In one embodiment, the front openings 214a and 214b, and the rear openings 216a and 216b are seam pockets. In one embodiment the front openings 214a and 214b, and the rear openings 216a and 216b comprise zippers to open and close the compartments 222a and 222b.

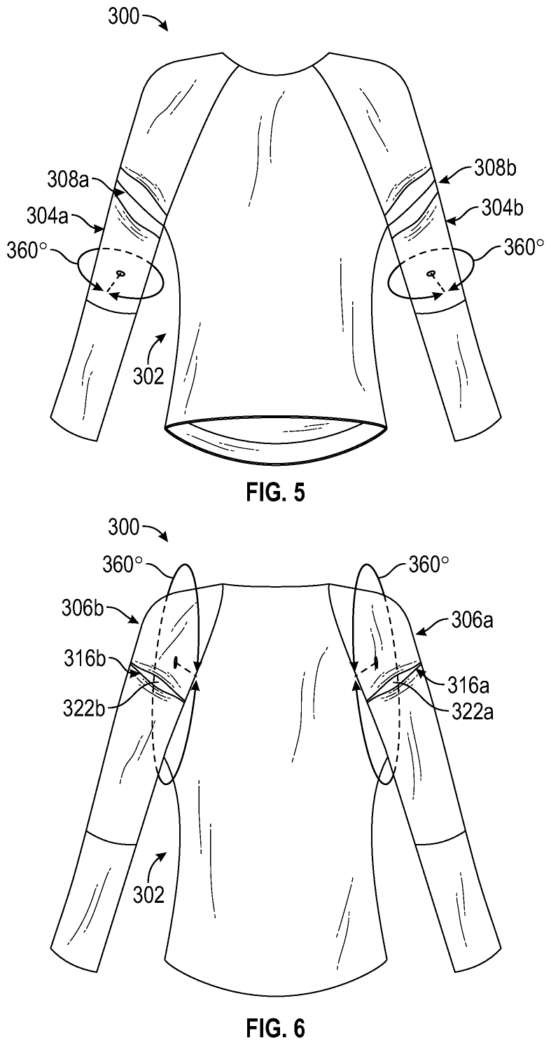

Referring now to FIGS. 5-6, in a different embodiment, a thermal therapy system 300 comprises a long sleeve compression shirt 302 with two elbow circular pouch shaped pockets 304a and 304b. In one embodiment, the elbow circular pouch shaped pocket 304a comprises a front opening 308a to open and close a compartment 312a (not shown), and the elbow circular pouch shaped pocket 304b comprises a front opening 308b to open and close a compartment 212b (not shown). In one embodiment, the compartments 312a and 312b are preferably functionally configured to receive and hold in place a thermal therapy device (not shown, but similar to the thermal therapy device 111 of FIG. 1), for example, a hot pack, a cold pack, and/or a hot/cold pack or combinations thereof. As denoted by the 360° arrows around the elbow areas of the long sleeve compression shirt 302 in FIG. 5, the versatility of the 360° elbow circular pouch shaped pockets 304a and 304b allows a user to selectively position one or more thermal therapy devices in the compartments 312a and 312b anywhere around the elbow of the user.

Referring more particularly to FIG. 6, in one embodiment, the thermal therapy system 300 also comprises two shoulder circular pouch shaped pockets 306a and 306b. In one embodiment, the shoulder circular pouch shaped pocket 306a comprises a rear opening 316a to open and close a compartment 322a, and the shoulder circular pouch shaped pocket 306b comprises a rear opening 316b to open and close a compartment 322b. In one embodiment, the compartments 222a and 222b are preferably functionally configured to receive and hold in place a thermal therapy device (not shown, but similar to the thermal therapy device 111 of FIG. 1), for example, a hot pack, a cold pack, and/or a hot/cold pack or combinations thereof. As denoted by the 360° arrows around the shoulder areas of the long sleeve compression shirt 302 in FIG. 6, the versatility of the 360° shoulder circular pouch shaped pockets 306a and 306b allows a user to selectively position one or more thermal therapy devices in the compartments 322a and 322b anywhere around the shoulder of the user.

In one embodiment, the front openings 308a and 308b, and the rear openings 316a and 316b are seam pockets. In one embodiment the front openings 308a and 308b, and the rear openings 316a and 316b comprise zippers to open and close the compartment 312a, 312b, 322a and 322b.

The various embodiments of the present invention have the advantage that they can accommodate one or more hot/cold packs in compartments that can comfortably hold the hot/cold packs in place and simultaneously provide a cloth barrier between the hot/cold packs and the person's skin.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalents.

### INDUSTRIAL APPLICABILITY

The embodiments of the invention are further illustrated by the following non-limiting example(s).

### Example 1

A pair of compression leggings with a waistband circular pouch shaped 360-degree pocket was manufactured out of a polyester and spandex blend fabric in the pattern shown in FIGS. 1-2 above. The waistband circular pouch shaped pocket had a front opening and a rear opening to access an interior compartment functionally configured to receive and hold in place a hot/cold pack. The compression material successfully held the pack in place over any area around the waist of the user (e.g., the lower abdomen, the lower back, the hips, appendix area, etc.). Users could place the hot/cold pack into the compartment through either the front opening or the rear opening. Users reported significant pain relief when using the leggings in conjunction with the hot/cold pack for 40-minute intervals. The versatility of the leggings allowed the users to remain active while applying heat or cold therapy to their waist. The hot/cold pack did not make direct contact with the skin of the users.

The preceding example can be repeated with similar success by substituting the generically or specifically described operating conditions, parameters, or elements of this invention for those used in the preceding example.

What is claimed is:

1. A thermal therapy system comprising:
a compression garment comprising an inner surface and an outer surface comprising one or more pockets;
said one or more pockets comprising each at least one opening and a circular pouch shape with an interior compartment functionally configured to receive and hold in place a thermal therapy pack.

2. The thermal therapy system of claim 1 wherein the interior compartment is functionally configured to receive and hold the thermal therapy pack over a target anatomical area.

3. The thermal therapy system of claim 2 wherein the target anatomical area is the user's waist.

4. The thermal therapy system of claim 2 wherein the target anatomical area is a limb of the user.

5. The thermal therapy system of claim 4 wherein the target anatomical area is the knee of the user.

6. The thermal therapy system of claim 4 wherein the target anatomical area is the ankle of the user.

7. The thermal therapy system of claim 4 wherein the target anatomical area is the elbow of the user.

8. The thermal therapy system of claim 4 wherein the target anatomical area is the shoulder of the user.

9. The thermal therapy system of claim 1 wherein the compression garment is a pair of leggings.

10. The thermal therapy system of claim 1 wherein the compression garment is a long sleeve shirt.

11. The thermal therapy system of claim 1 wherein the thermal therapy pack is a hot pack.

12. The thermal therapy system of claim 1 wherein the thermal therapy pack is a cold pack.

13. The thermal therapy system of claim 1 wherein the thermal therapy pack is a hot/cold pack.

14. The thermal therapy system of claim 1 wherein the one or more pockets are seam pockets.

15. The thermal therapy system of claim 14 wherein the one or more pockets are seam pockets comprising zippers to open and close.

16. A thermal therapy garment system comprising:
a compression garment comprising an inner surface and an outer surface comprising one or more pockets;
said one or more pockets comprising each at least one opening and a circular pouch shape with an interior compartment functionally configured to receive and hold in place a thermal therapy pack over a target anatomical area.

17. The thermal therapy garment system of claim 16 wherein the target anatomical area is the user's waist.

18. The thermal therapy garment system of claim 16 wherein the target anatomical area is a limb of the user.

19. The thermal therapy garment system of claim 16 wherein the compression garment is a pair of leggings.

20. A thermal therapy garment system comprising:
a pair of compression fabric leggings comprising an inner surface and an outer surface comprising a circular pouch shaped pocket on the waist band; and
the circular pouch shaped pocket comprises a front opening and a rear opening and an interior compartment functionally configured to receive and hold in place a thermal therapy pack anywhere over the waist of a user.
